(19) United States
(12) Patent Application Publication
(10) Pub. No.: US 2007/0021634 A1
(43) Pub. Date: Jan. 25, 2007

(54) SYNTHESIS OF THERAPEUTIC DIPHENYL ETHERS

(75) Inventors: Yong Tao, Salem (CA); Daniel W. Widlicka, Gales Ferry, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/458,035

(22) Filed: Jul. 17, 2006

Related U.S. Application Data
(60) Provisional application No. 60/700,900, filed on Jul. 19, 2005.

(51) Int. Cl. C07C 209/44; C07C 235/32

(52) U.S. Cl. 564/414; 564/165

(57) ABSTRACT

This invention is directed to a method of synthesizing compounds of Formula I and to intermediates useful in the synthesis of compounds of Formula I 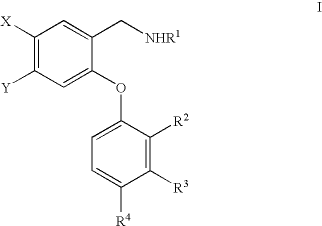
wherein X, Y, R¹, R², R³, and R⁴ are as defined herein above.

### FIELD OF THE INVENTION

The present invention provides a novel synthesis of diphenyl ether compounds which are useful for the treatment of depression and other disorders.

### BACKGROUND OF THE INVENTION

U.S. Ser. No. 60/608994, the contents of which are incorporated herein by reference, discloses a procedure for making a specific example of the compound of Formula I, namely Formula IA, which is depicted in Scheme I below. The procedure in Scheme I affords the compound of Formula IA in an overall 15% yield. A significant amount of the 4-substituted side product, greater than 10%. was formed in the reaction between the aldehyde and the phenol.

### SUMMARY OF THE INVENTION

One aspect of the invention relates to a novel method of making a compound of Formula I: 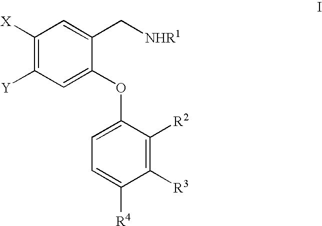
or a pharmaceutically acceptable salt or solvate thereof wherein:

X is H, halo, or CH₃;

Y is halo, (C₁-C₆)alkyl, —CR⁵R⁶—(CH₂)ₙCH₃, or —S(O)ₘ—(CH₂)ₚCH₃;

m is 0, 1, or 2;

n is 0, 1, 2, 3, 4, 5, or 6;

p is 0, 1, 2, 3, 4, 5, or 6;

r is 0, 1, 2, 3, 4, 5, or 6;

s is 0, 1, 2, 3, 4, 5, or 6;

t is 0, 1, 2, 3, 4, 5, or 6;

R¹ is H or (C₁-C₆)alkyl;

R² is H, halo, —O(CH₂)ᵣCH₃, (C₁-C₆)alkyl, or CN,

R³ is H, halo, (C₁-C₆)alkyl, —O—(CH₂)ₛCH₃, Cl, CN, —N(R⁷)(R⁸), or OH;

R⁴ is H, halo, (C₁-C₆)alkyl, —O—(C₁-C₆)alkyl; —S—(C₁-C₆)alkyl; OH, —NH—R⁹, or —S(O)ᵣ—(C₁-C₆)alkyl; and

R⁵, R⁷, R⁸, and R⁹ are each independently selected from H or (C₁-C₆)alkyl,

wherein said method comprises the steps of:

i) reacting a compound of Formula A with NH₂R¹ to form a compound of Formula B:

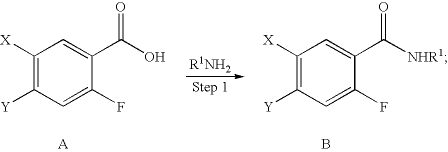

ii) reacting the compound of Formula B with a compound of Formula C to form a compound of Formula D:

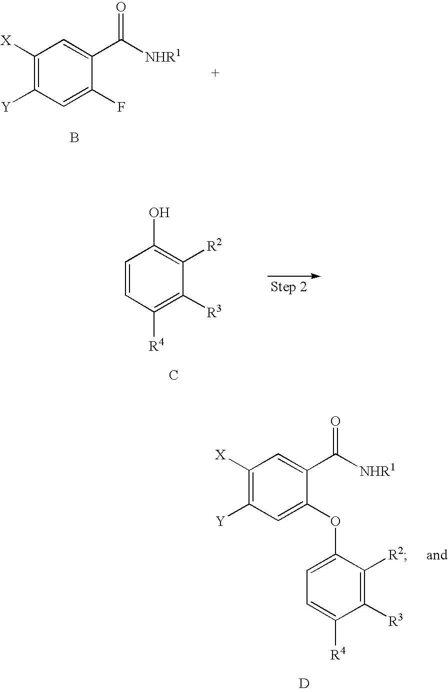

iii) reducing the compound of Formula D with a reducing agent to form the compound of Formula I.

Another aspect of the invention relates to a compound of Formula B: 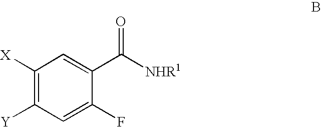
wherein X, Y, and R¹ are as defined above Compound B is an intermediate particularly useful for preparing the compound of Formula I.

Another aspect of the present invention relates to a compound of Formula D: 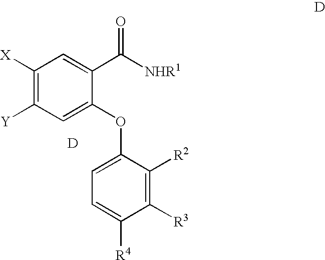
or a pharmaceutically acceptable salt or solvate thereof wherein X, Y, R¹, R², R³ and R⁴ are as defined above. Compound D is an intermediate also particularly useful for preparing compounds of Formula I.

Another aspect of the invention relates to radiolabeled compounds of Formulae B and D. Such radiolabeled compounds are useful as intermediates for preparing radiolabeled compounds of Formula I. Radiolabeled compounds of Formula I are useful as imaging agents and biomarkers for medical therapy and diagnosis and as pharmacological tools for studying 5HT function and activity.

### DETAILED DESCRIPTION OF THE INVENTION

The present invention relates methods of making certain diphenyl ethers which are ligands for 5HT₂, receptors, and to intermediates which are useful in the preparation of those diphenyl ethers.

One aspect of the invention relates to an improved method of making compounds of Formula I: 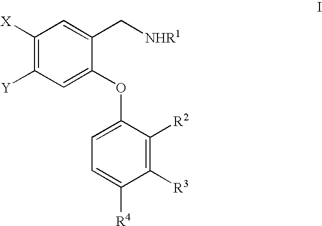
wherein X, Y, R¹, R², R³, R⁴ are as defined herein above.

In a preferred embodiment, the compound of Formula A is reacted with (C₁-C₆)alkyl-OH prior to adding NH₂R¹to form intermediate compound B(i) as shown below in Scheme 3A:

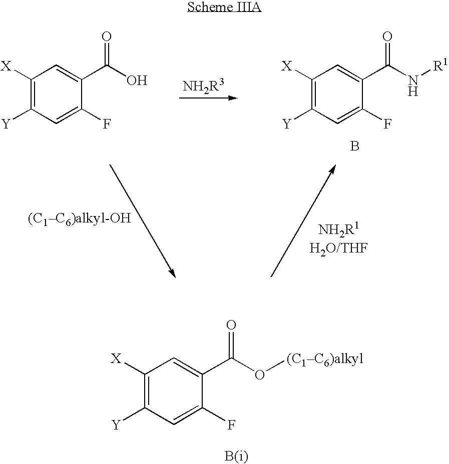

In another preferred embodiment, X is halo, Y is halo, R¹ is (C₁-C₆)alkyl, R² is (C₁-C₆)alkyl, R³ is —O—(CH₂) ₛCH₃, and R⁴ is H for the compound of Formula I.

Unless otherwise indicated, the following terms and related variations of the same as used herein representatively have the meanings ascribed:

The term “alkyl,” as used herein, unless otherwise indicated, includes saturated monovalent hydrocarbon radicals with 1-12 carbon atoms having straight, branched or cyclic moieties or combinations thereof. The term “lower alkyl” refers to an alkyl group having one to six carbon atoms, Examples include methyl, ethyl, propyl, isopropyl, cyclopropyl, butyl, isobutyl, sec-butyl, t-butyl, pentyl, neopentyl, cyclopentylmethyl, and hexyl. It is preferred that the alkyl group is lower alkyl. The preferred cyclic alkyl groups are cyclobutyl and cyclopentyl. The preferred lower alkyl group contains 1-3 carbon atoms.

The term “halo” or “halogen” as used herein, unless otherwise indicated, includes F, Cl, Br, and I Chlorine and fluorine are preferred. Alkyl groups substituted with one or more halogen atoms include chloromethyl, 2,3-dichloropropyl, and trifluoromethyl. It is preferred that the halo groups are the same. The most preferred halogen-substituted alkyl group is trifluoromethyl.

The chemist of ordinary skill will recognize that certain combinations of substituents included within the scope of Formula (I) may be chemically unstable. The skilled chemist will either avoid these combinations or protect sensitive groups with well-known protecting groups. As used herein, the term “deprotecting” refers to the removal of such well-known protecting groups by methods that are well known in the art.

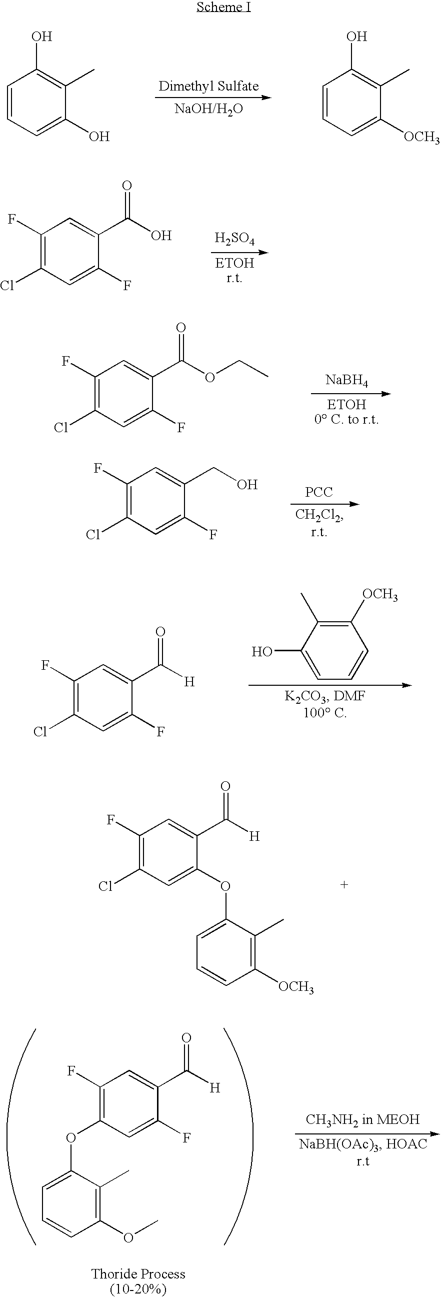

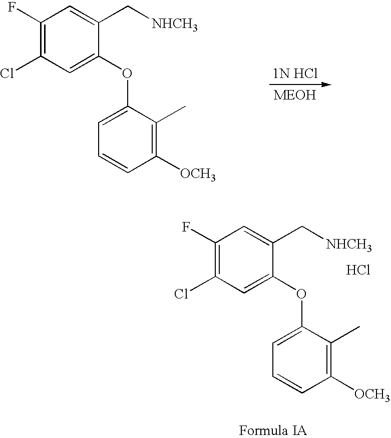

The process of making the specific compound of Formula A in the present invention is exemplified in Scheme II below. Scheme II improves upon the earlier synthesis in both the number of steps and the regioselectivity. This novel method proceeds via an amide intermediate.

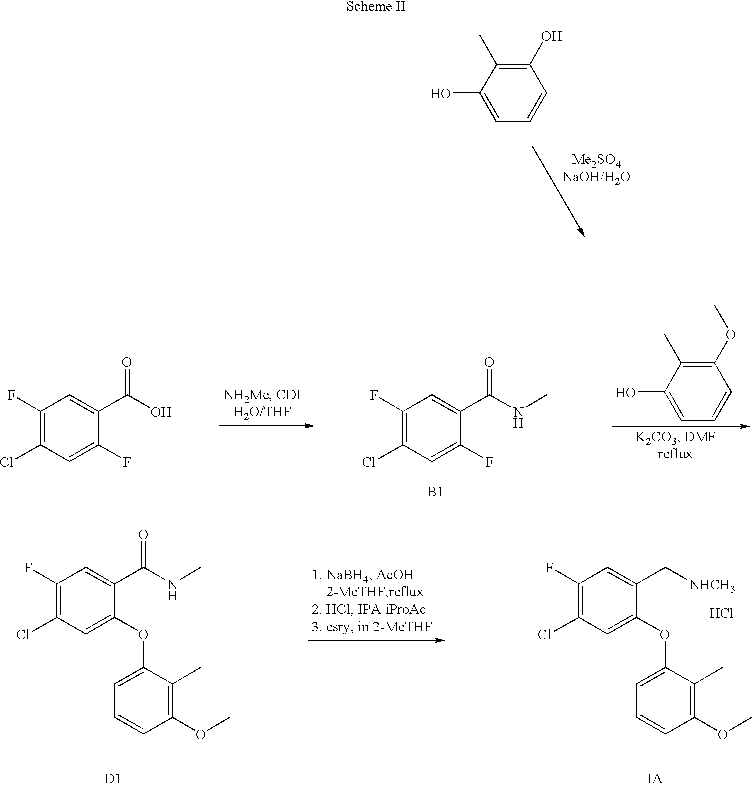

The amide intermediate compound of Formula B1, N-methyl 2,5-difluoro-4-chloro-benzamide, can be synthesized from the commercial acid as shown in Scheme IIIB using either a one-step procedure or a two-step procedure. Both methods afforded Compound B1 in high yields.

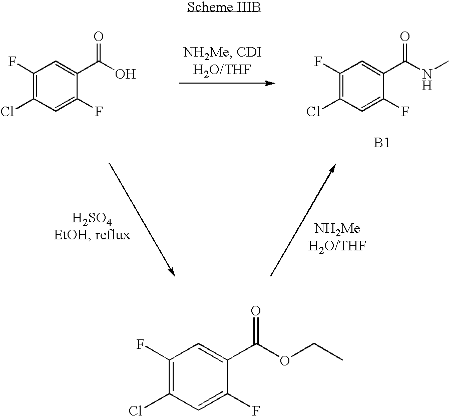

The product from Scheme III, the compound of Formula B1, is precipitated from the reaction mixture after the addition of water.

As shown above in Scheme II, the side chain, 2-methyl-3-methoxy-phenol, was prepared through methylation of 2-methyl resorcinol with dimethyl sulfate in aqueous sodium hydroxide solution. When most of the starting material had been consumed, the di-methoxy side product was removed by extraction with ether. The crude material was extracted with dichloromethane after acidification. Passing a short silica gel pad afforded purified 2-methyl-3 -methoxy-phenol at 53% yield.

Conditions for ether formation were screened. The results are shown in Table 1 below.

TABLE 1

| | | Solvent | Reaction | | Conver- | |
|---|---|---|---|---|---|---|
| Entry | Base | (Reflux) | Time | | sion | Yield |
| 1 | K₂CO₃ (2 eq) | DMF | 12 | h | 90% | 62.5% |
| 2 | K₂CO₃ (2 eq) | DME | 6 | d | 26% | NA |
| 3 | K₂CO₃ (2 eq) | NMP | 18 | h | 88% | NA |
| 4 | tBuOK (1.1 eq) | THF | 6 | d | 82% | NA |
| 5 | tBuOK (1.1 eq) | 2-MeTHF | 5 | d | 90% | 61% (pure) |
| 7 | tBuOK (1.1 eq) | Toluene | 4 | d | 93% | NA |

Unlike the intermediate aldehyde of Scheme I, the amide (Compound B1) in Scheme II afforded almost mostly the desired compound of Formula IA under all of the above tested conditions. The reaction was complete in several hours with potassium carbonate in refluxing DMF and required several days to reach completion with potassium t-butoxide in refluxing 2-methyl tetrahydrofuran. The product, 4 chloro-5-fluoro-2-(3-methoxy-2-methyl-phenoxy)—N—methyl-benzamide (Compound D1), was isolated by recrystallization from isopropyl ether.

A suitable condition to reduce the amide to the target amine, Formula IA, ,was developed, Treatment of the amide with LAH in refluxing tetrahydrofuran or 2-methyl tetrahydrofuran resulted in a mixture of the desired product with 10 to 20% of the dechlorinated side product, which was difficult to remove. Treatment with a freshly prepared borane, which was generated in situ from boron trifluoride diethyl etherate and sodium borohydride, drove the reaction to 80% conversion. A procedure using sodium acetoxyborohydride (5 eq ), reported in Vogel's textbook of Practical Organic Chemistry, was employed and gave a satisfactory result. The reducing agent was generated in situ from sodium borohydride and acetic acid and the reaction reached completion cleanly in refluxing 2-methyl tetrahydrofuran in 6 hours. The reaction also went well in other refluxing ethereal solvents, such as dimethoxyethane, 1,4-dioxane and tetrahydrofuran. Decreasing the amount of sodium acetoxyborohydride to 2.5 eq. resulted in 70%. conversion. The product-borane complex was broken by a 2-hour reflux in a mixture of 2N aq. hydrochloric acid and 2-methyl tetrahydrofuran (5:1) The HCl salt of Formula IA precipitated when the mixture was cooled. After filtration, [4-chloro5-fluoro-2-(3methoxy-2-methyl-phenoxy)-benzyl]methylamine hydrochloride, Formula I, was obtained at 83% yield.

Another aspect of the invention relates to a compound of Formula B: 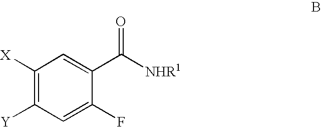
or a pharmaceutically acceptable salt or solvate thereof, wherein X, Y, and R¹ are as defined above. Compound B is an intermediate particularly useful for preparing the compound of Formula (I). A specific example of the compound of Formula B is the compound of Formula B1: 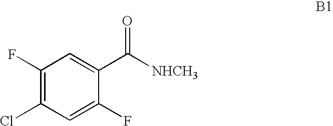
Another aspect of the present invention relates to a compound of Formula D: 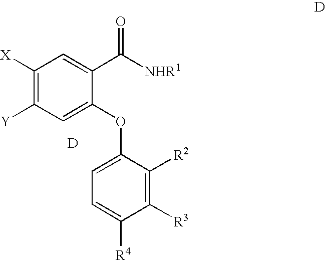
or a pharmaceutically acceptable salt or solvate thereof, wherein X, Y, R¹, R², R³ and R⁴ are as defined above, Compound D is an intermediate also particularly useful for preparing compounds of Formula (I.) A specific example of the compound of Formula D is the compound of Formula D1.

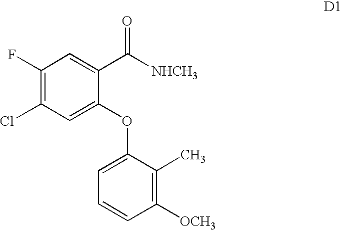

Another aspect of the invention relates to radiolabeled compounds of Formulae (B) and (D). Radiolabeled compounds of Formulae (B) and (D) can be prepared by incorporation into the synthetic procedures described hereinabove of techniques of isotopic labeling that are well known in the art. Any radioisotope capable of being detected can be employed as a label. A compound is radiolabeled either by substitution of a radioactive isotope of hydrogen, carbon, or fluorine or by incorporation of a phenyl group that is substituted with radioactive iodine. Suitable radioisotopes include carbon-11, fluorine-18, fluorine-19, iodine-123 and iodine-125. For example, see Arthur Murry III, and D. Lloyd Williams, “Organic Synthesis with Isotopes,” vols. I and II. Interscience Publishers Inc., N.Y, (1958) and Melvin Calvin et al. “Isotopic Carbon,” John Wiley and Sons Inc., N.Y. (1949). Preferably, a compound of Formula (I) may be labeled by adding one or more radioisotopes of a halogen (e.g. iodine-123) to an aromatic ring, or by alkylating a nitrogen atom in a compound of Formula (I) with a group comprising a phenyl group bearing a radioisotope.

In the examples below the following terms are intended to have the following, general meaning:

OMF dimethyformamide

DMA: dimethyl acetamide

cm: centimeter

doublet: (spectral)

EPS: extrapyramidal syndrome

g: grams

GC: gas chromatography

J: coupling constant (in NMR)

L: liter(s)

LAH: lithium aluminum hydride

LC: liquid chromatography

ml: milliliter

mmol: millimole.

Mp: melting point

MS: Mass Spec.

(NMR): nuclear magnetic resonance

q, quartet

rt: room temperature

s: singlet

t: triplet

THF: tetrahydrofuran

The following examples are illustrative only; they are not restrictive

### EXAMPLE 1

### N-methyl 2,5-difluoro-4chloro-benzamide (One Step)

40% Methylamine in water (54 ml, 624 mmoles) was added dropwise to a mixture of 2.5-difluoro-4-chloro-benzoic acid (PLEASE DEFINE THE SOURCE OF THIS REACTANT) (30.0 g, 155.8 mmoles) and 1,1′-carbonyldiimidazole (27.6 g, 170 mmoles) in THF (300 ml). The mixture vas stirred at room temperature for 40 minutes. Water (1.8L) was added to the reaction mixture. The slurry was stirred at room temperature for 30 minutes and filtered. The cake was washed with water and dried in a vacuum oven at 40° C. for 18 hours. N-methyl 2,5-difluoro-4-chloro-benzamide (26.5 g) was obtained at 82.7% yield.

Mp: 127.5-127.6° C. ¹H NMR (in CDCl₃): δ7.88 (dd, J=9.5/7.0, 1H), 7.20 (dd, J=10.4/5.4, 1H), 6.67 (brs, 1H), 3.00 (d, J=4.5, 3H). GC-MS: 205 (M+).

### EXAMPLE 2

### Ethyl2,5-difluoro-4-chloro-benzoate

Sulfuric acid (62.4 ml, 1.13 moles) was added to a solution of 2,5-difluoro-4-chloro-benzoic acid (750.0 g, 3.895 moles) (PLEASE DEFINE THE SOURCE OF THIS REACTANT) in absolute ethanol (3.75 L). The mixture was refluxed for 18 hours. The mixture was concentrated to remove most of ethanol and then cooled to room temperature. The residue was neutralized with 1N aqueous NaOH (2.4 L) and extracted with ethyl acetate (2×1L). The combined extracts were washed with sat. aqueous NaHCO₃ and brine, then dried with (MgSO₄) and concentrated to dryness. Ethyl 2,5-difluoro-4-chloro-benzoate (806.7 g) was obtained at 94% yield.

¹H NMR (in CDCl₃): δ7.72 (dd, J=9.1/6.2, 1H), 7.22 (dd, J=9.1/5.6, 1H), 4.39 (q, 3J=7.0, 2H), 1.39 (t, J=7.0, 3H). GC-MS: 161 (M+).

### EXAMPLE 3

### N-methyl 2,5-difluoro-4-chloro-benzamide (Two Steps)

40% methylamine in water (303 ml. 3.5 moles) was added to a mixture of ethyl 2.5-difluoro-4-chloro-benzoate (154 g, 0.7 moles) (prepared from Example 2) in THF (250 ml) and water (250 ml). The mixture was stirred at room temperature for 2 hours. Water (500 ml) was added and most of the THF was distilled off. The resulting slurry was stirred at room temperature for 1 hour and filtered. The cake was washed with water and dried in a vacuum oven at 40° C. for 16 hours. N-methyl 2,5-difluoro-4-chloro-benzamide (132.6 g) was obtained at 92.4% yield.

Mp: 127.5-127.6° C. ¹H NMR (in CDCl₃): δ7.88 (dd, J=9.5/7.0. 1H), 7.20 (dd, J=10.4/5.4, 1H), 6.67 (brs), 1H), 3.00 (d, J=4.5, 3H). GC-MS: 205 (M+).

### EXAMPLE 4

### 2-Methyl-3-methoxyphenol

A solution of 2-methyl resorcinol (550 g, 4.43 mole) (PLEASE DEFINE THE SOURCE OF THIS REACTANT) and sodium hydroxide (212 g, 5.30 mole) in water (2.65 l) was heated to reflux. Dimethyl sulfate (462 ml, 4.88 mole) was added via an addition funnel over a period of 45 minutes. The mixture was refluxed for 30 minutes. 10% wt. aqueous sodium hydroxide (2 L) was added to the reaction mixture. The mixture was stirred at 80° C. for 10 minutes and then cooled to room temperature. The mixture was extracted with diethyl ether (2 L). Hydrochloric acid (36% in water, 800 ml) was added slowly to adjust the pH of the mixture below 2. The mixture was extracted with dichloromethane (3 L plus 2×700 ml). The combined dichloromethane extracts were washed with water (1 L), dried (MgSO₄) and concentrated The residue was passed a pad of silica gel (1 kg, 16×14 cm) and washed with 1:1 hexane-dichloromethane. After the concentration of the fractions, 2-methyl-3 methoxyphenol (324 g) was obtained at 53% yield.

¹H NMR (in CDCl₃): δ7.00 (t, J=8.3, 1H), 6.43 (t, J=8.3, 1H), 4.68 (s, 1H), 3,79 (s, 3H), 2.09 (s, 3H). GC-MS: 138 (M+).

### EXAMPLE 5

### 4-chloro-5-fluoro-2-(3-methoxy-2-methyl-phenoxy)-N-methyl-benzamide

### Method 1 (in DMF)

A suspension of 2-Methyl-3-methoxyphenol (12.42 g, 89.9 mmol) (prepared from Example 4), N-methyl 2,5-difluoro-4-chloro-benzamide (18.48 g, 89.9 mmol) (prepared from Example 1 or Example 3) and potassium carbonate (Aldrich, <325 mesh, 27.33 g, 197.7 mmol) in DMF (170 ml) was stirred at 110° C. for 12 hours, The reaction mixture was cooled to room temperature. Water (680 ml) was added slowly and followed by isopropyl ether (150 ml). The slurry was stirred at room temperature for 2 hours and filtered. The cake was washed with water (100 ml) and isopropyl ether (50 ml) and dried in a vacuum oven (40° C.) for 16 hours. 4 chloro-5fluoro-2-(3-methoxy-2-methyl-phenoxy)-N-methyl-benzamide (18.18 g) was obtained as a beige granule at 62.5% yield.

Mp: 126.7-128.1° C. ¹H NMR (in CDCl₃): δ8.05 (d, J=9.9, 1H), 7.77 (brs, 1H), 7.21 (t, J=8.3, 1H), 6.77 (d, J=8.3, 1H), 6.59 (m, 2H), 3.86 (s, 3H), 2.99 (d, J=3.8, 3H), 2.04 (s, 3H). LC-MS: 324.4 (ES+).

### Method 2 (in 2-methyl tetrahydrofuran)

A suspension of 2-Methyl-3-methoxyphenol (241.8 g, 1.75 mole) (prepared from Example 4), N-methyl-2,5-difluoro-4-chloro-benzamide (342.6 g, 1.67 mole) (prepared from Example 1 or Example 3) and potassium t-butoxide (196.4 g, 1.75 mole) in 2-methyltetrahydrofuran (3.4 L) was refluxed for 120 hours. The reaction mixture was cooled to room temperature. Water (1.5 L ml), brine (1.5 L) and 1N aqueous hydrochloric acid (900 ml) were added to the reaction mixture. The organic layer was isolated and the aqueous layer was extracted with 2-methyltetrahydrofuran (2×1 L). The combined organic extracts were washed with brine (1.5 L), dried (Mg₂SO₄) and concentrated to dryness. Isopropyl ether (1.8 L) was added to the residual solid. The mixture was heated to reflux and stirred under reflux for 1 hour. The mixture was cooled to 5° C. The slurry was stirred for 2 hours at 5° C. and filtered. The cake was washed with isopropyl ether (3×100 ml and dried in a vacuum oven (40° C.) for 16 hours. 4-chloro-5-fluoro-2-(3-methoxy-2-methyl-phenoxy)-N-methyl-benzamide (333.2 g) was obtained at 61% yield.

Mp: 126.7-128.1° C. ¹H NMR (in CDCl₃): δ8.05 (d, J=9.9, 1H), 7.77 (brs, 1H), 7.21 (t, J=8.3, 1H), 6.77 (d, J=8.3, 1H), 6.59 (m, 2H), 3.86 (s, 3H), 2.99 (d, J=3.8, 3H), 2.04 (s, 3H). LC-MS: 324.4 (ES+).

### EXAMPLE 6

### [4-chloro-5-fluoro-2-(3-methoxy-2-methyl-phenoxy)-benzyl]methylamine hydrochloride

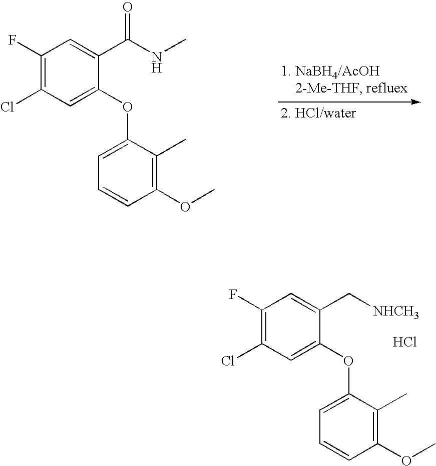

Sodium borohydride (5.82 g, 150 mmol) was added to a solution of 4-chloro5-fluoro-2-(3-methoxy-2-methyl-phenoxy)-N-methyl-benzamide (10.0 g, 30.9 mmol) in 2-methyl tetrahydrofuran (70 ml). The suspension was cooled to 2° C. A solution of acetic acid (8.8 ml, 150 mmol) in 2-methyl tetrahydrofuran (30 ml) was added over a period of 20 minutes The mixture was heated to reflux and the reflux lasted for 22 hours. The mixture was cooled to 15° C. and poured to a stirred mixture of water and ice (140 ml), 25% wt. aqueous sodium hydroxide (10 ml) was added and the mixture was extracted with 2-methyl tetrahydrofuran (2×60 ml), The combined organic extracts were washed with brine 9100 ml) and concentrated to dryness. 2-methyl tetrahydrofuran (20 ml) and 2N aqueous hydrochloric acid (100 ml) were added to the residual oil. The mixture was refluxed for 2 hours and then cooled to room temperature. The slurry was filtered. The cake was washed with water (5 ml) and 2-methyl tetrahydrofuran (3×10 ml) and dried in a vacuum oven (40° C.) for 24 hours. [4-chloro5-fluoro-2-(3-methoxy-2-methyl-phenoxy)-benzyl]methylamine Hydrochloride, (8.86 g) was obtained at 83% yield.

Mp: 189.3-190.3° C. ¹H NMR (in CD₃OD): δ7.44 (d, J=9.1, 1H), 7.23 (t, =8.3, 1H), 6.88 (d, J=8.3, 1H), 6.61 (m, 2H), 4.32 (s, 2H), 3.86 (s, 3H), 2.77 (s, 3H), 2.03 (s, 3H). LC-MS: 310.4 (ES+).

| | Elemental Analysis: | | |
|---|---|---|---|
| | C % | H % | N % |
| Calculated: | 55.50 | 5.24 | 4.05 |
| Found: | 55.58 | 5.16 | 3.96 |

Based on a reading of the present description and claims, certain modifications to the methods and compounds described herein will be apparent to one of ordinary skill in the art. The claims appended hereto are intended to encompass these modifications.

1. A method for preparing a compound of Formula I having the structure: 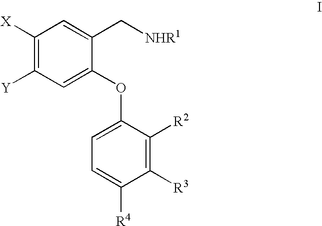
or a pharmaceutically acceptable salt or solvate thereof, wherein;
X is H, halo, or CH₃;
Y is halo, (C₁-C₆)alkyl, —CR⁵R⁶—(CH₂)ₙCH₃, or —S(O)ₘ—(CH₂)ₚCH₃;
m is 0, 1, or 2;
n is 0, 1, 2, 3, 4, 5, or 6;
p is 0, 1, 2, 3, 4, 5, or 6;
r is 0, 1, 2, 3, 4, 5, or 6;
s is 0, 1, 2, 3, 4, 5, or 6;
t is 0, 1, 2, 3, 4, 5, or 6;
R¹ is H or (C₁-C₆)alkyl;
R² is H, halo, —O(CH₂)ᵣCH₃, (C₁-C₆)alkyl, or CN;
R³ is H, halo, (C₁-C₆)alkyl, —O—(CH₂)₅CH₃, Cl, CN, —N(R⁷)(R⁸), or OH;
R⁴ is H, halo, (C₁-C₆)alkyl, —O—(C₁-C₆)alkyl; —S—(C₁-C₆)alkyl; OH, —NH—R⁹, or —S(O)ₜ—(C₁-C₆)alkyl; and
R⁶, R⁷, R⁸, and R⁹ are each independently selected from H or (C₁-C₆)alkyl; wherein said method comprises the steps of:
ii) reacting a compound of Formula A with NH₂R¹ to form a compound B: 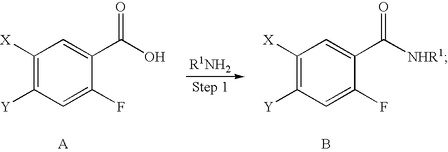
ii) reacting the compound of Formula B with a compound of Formula C to form a compound of Formula D; 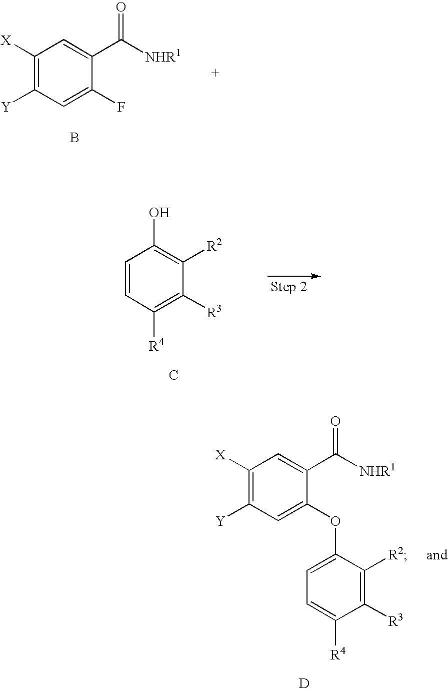
iii) reducing the compound of Formula D with a reducing agent to form the compound of Formula I.

2. The method according to claim , wherein Step 1 further comprises reacting the compound of Formula A with (C₁-C₆)alkyl-OH prior to adding NH₂R¹ to form intermediate compound B(i);

3. The method according to claim 1, wherein X is halo, Y is halo, R¹ is (C₁-C₆)alkyl, R² is (C₁-C₆)alkyl, R³ is —O—(CH₂)ₛCH₃, and R⁴ is H.

4. The method according to claim 1, wherein the compound of Formula I formed is Formula IA 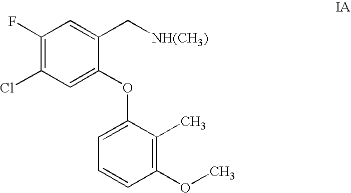
or a pharmaceutically acceptable salt or solvate thereof.

5. The method according to claim 1, wherein the reducing agent used in step (iii) is LAH or acetoxyborohyrdride.

6. The method according to claim 5 wherein the reducing agent used in step (iii) is acetoxyborohyrdride.

7. A compound having Formula B: 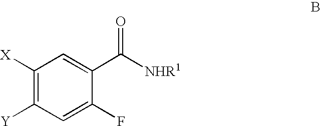
or a pharmaceutically acceptable salt or solvate thereof wherein:
X is H, halo, or CH₃;
Y is halo, (C₁-C₆)alkyl, —CR⁵R⁶—(CH₂)ₙCH₃, or —S(O)ₘ—(CH₂)ₚCH₃); and
m is 0, 1, or 2;
n is 0, 1, 2, 3, 4, 5, or 6;
p is 0, 1, 2, 3 4, 5, or 6; and
R¹, R⁵ and R⁶ are each independently selected from H or (C₁-C₆)alkyl.

8. The compound according to claim 7 having Formula B1: 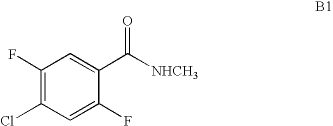
or a pharmaceutically acceptable salt or solvate thereof.

9. A compound having Formula D: 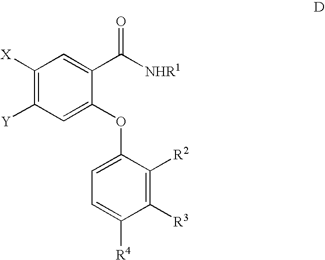
or a pharmaceutically acceptable salt or solvate thereof, wherein:
X is H, halo, or CH₃;
Y is halo, (C₁-C₆)alkyl, —CR⁵R⁶—(CH₂)ₙCH₃, or —S(O)ₘ—(CH₂)ₚCH₃;
m is 0, 1, or 2;
n is 0, 1, 2, 3, 4, 5, or 6;
p is 0, 1, 2, 3, 4, 5or 6;
r is 0, 1, 2, 3, 4, 5, or 6;
s is 0, 1, 2, 3, 4, 5, or 6;
t is 0, 1, 2, 3, 4, 5, or 6;
R¹ is H or (C₁-C₆)alkyl;
R² is H, halo, —O(CH₂), CH₃, (C₁-C₆)alkyl, or CN;
R³ is H, halo, (C₁-C₆)alkyl, —O—(CH₂)ₛCH₃, Cl, CN, —N(R⁷)(R⁸), or OH;
R⁴ is H, halo, (C₁-C₆)alkyl, —O—(C₁-C₆)alkyl; OH, —NH—R⁹, or —S(O)ₜ—(C₁-C₆)alkyl; and
R⁶, R⁷, R⁸, and R⁹ are each independently selected from H or (C₁-C₆)alkyl.

10. The compound according to claim 9 having Formula D1: [chemical structure]
